(19) United States
(12) Patent Application Publication
(10) Pub. No.: US 2026/0069548 A1
(43) Pub. Date: Mar. 12, 2026

(54) Systems and Methods of Exosome Mediated Delivery of Antibodies

(71) Applicant: Synaptec Network, Inc., Santa Monica, CA (US)

(72) Inventor: Sheldon Jordan, SANTA MONICA, CA (US)

(21) Appl. No.: 19/242,348

(22) Filed: Jun. 18, 2025

Related U.S. Application Data
(60) Provisional application No. 63/661,312, filed on Jun. 18, 2024.

(51) Int. Cl. A61K 9/50; A61K 38/17; A61K 41/00

(52) U.S. Cl. CPC A61K 9/5068; A61K 38/1774; A61K 41/0047

(57) ABSTRACT

Methods and systems for improving or enabling delivery of antibodies, immunoglobulin receptors, or immunotherapy agents across the Blood-Brain Barrier (BBB) are disclosed. Exosomes are derived from stem cells, immune cell progenitors, or immune cells, and are loaded with an antibody, immunoglobulin receptor, or antibody fragment. Focused energy, preferably low-intensity focused ultrasound, is applied to a region of the BBB of a patient, and loaded exosomes are administered to the patient. Exosomes target the region for delivery of the loaded antibody, receptor, or fragment, either by traversing the BBB at the region or releasing the loaded antibody to neural tissue at the region.

This application claims the benefit of priority to United States Provisional Patent No. 63/661,312 filed Jun. 18, 2024, which is incorporated by reference in their entirety herein.

### FIELD OF THE INVENTION

The field of the invention is treatment of neurological conditions.

### BACKGROUND

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

It is increasingly desirable to enable or improve delivery of therapeutic agents across the Blood-Brain Barrier (BBB). The BBB prevents foreign elements from entering the brain, and often prevents objects from entering the brain that may have therapeutic effect. For example, antibodies, antibody fragments, immunoglobin, or immunoglobin fragments generally will not natively cross the BBB to enter the brain. While it is known to mechanically bypass the BBB with a needle or laceration, this is invasive and can cause unnecessary or unacceptable damage to a patient.

Less invasive methods for crossing the BBB are known. For example, “Targeted Delivery of Neural Stem Cells to the Brain Using MRI-Guided Focused Ultrasound to Disrupt the Blood-Brain Barrier,” PLOS ONE 6(11): e27877 by Burgess, et al., reports use of focused ultrasound with microbubbles to open targeted regions of the BBB in animal models. “Cellular Mechanisms Of The Blood-Brain Barrier Opening Induced By Ultrasound In Presence Of Microbubbles,” Ultrasound in Med. & Biol., Vol. 30, No. 7, pp. 979-989, 2004 by Sheikov, et al., reports use of ultrasound and microbubbles in animal models to open the BBB, noting the BBB can be damaged at 3W or greater sonication. “Noninvasive Localized Delivery Of Herceptin To The Mouse Brain By Mri-Guided Focused Ultrasound-Induced Blood-Brain Barrier Disruption,” PNAS, Vol. 103, No. 31, 11719-23 by Kinoshita, et al., reports use of ultrasound with microbubbles in animal models to disrupt BBB, noting microbubbles are required for consistent BBB opening. US Patent Publ. 2020/0108241 to Jordan, et al., also teaches use of ultrasound to improve delivery of exosomes across the BBB.

Thus, there remains a need for systems and methods to improve delivery of antibodies, immunoglobulin receptors, or other immune agents or immunologics across the BBB without damaging the BBB.

### SUMMARY OF THE INVENTION

Methods of delivering an immune agent (e.g., an immunoglobulin (Ig) receptor, Ig fragment, whole or partial Ig, whole of partial antibody, etc.) across a blood-brain barrier (BBB) of a patient include loading an exosome with the immune agent. A region of the BBB is stimulated to improve delivery of the immune agent or immunoglobulin receptor across the BBB at the region. The exosome is introduced to the region of the BBB and the immune agent or immunoglobulin receptor from the exosome is exposed to neural tissue of the patient, for example after crossing the BBB.

Methods of delivering an immune component (e.g., immunoglobulin (Ig) component, Ig receptor, Ig fragment, whole or partial Ig, whole or partial antibody, etc.) across the BBB of a patient are contemplated. An exosome having (e.g., carrying, embedding, encasing, expressing, etc.) the immune component is administered to the patient. Focused ultrasound, preferably low-intensity focused ultrasound, is applied to a region of the BBB, and the immune component is exposed to neural tissue of the patient. The immune component can be exposed or applied to the region by the exosome traversing the BBB at the region and releasing the immune component. Alternatively or in combination the exosome can release the immune component through the BBB without the exosome itself passing through the BBB.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 depicts a method of the inventive subject matter.

FIG. 2 depicts another method of the inventive subject matter.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

Methods and systems are contemplated for use of exosomes derived from immune cells or cells known to have receptors for immunoglobulins (e.g., monocytes or similar cell lines), which may be sourced from a patient's own cells (autologous) or from another source (allogeneic). A low-intensity focused ultrasound technique (e.g., without microbubbles) is directed at the BBB and such exosomes are administered to the patient. The combination of such exosomes with low-intensity focused ultrasound technique increases adhesion of the exosomes to the BBB without disruption or compromising the structural integrity of the BBB.

In some embodiments, an exosome is derived from either a cell line that is known to have Ig receptors (e.g., monocytes, leukocytes, lymphocytes, dendritic cells, T/B cells, etc.) or an otherwise generally-sourced exosome that is engineered or altered to carry an IG receptor. The exosome is co-incubated or otherwise mixed or loaded with an immune agent or component (e.g., whole or partial Ig, Ig fragment, antibody fragment, etc.). It is contemplated the immune agent itself is of a type or quality that it cannot cross the BBB natively or without intervention.

The exosome containing the immune agent can traverse the BBB. Such exosomes can also be targeted or have improved delivery to or through the BBB by use of focused ultrasound, light therapy, TMS, shockwave, microwave, or other methods of applying focused energy to the BBB.

Systems and methods of delivering an immune component or agent or an immunoglobulin receptor across a blood-brain barrier (BBB) of a patient include loading an exosome with the immune agent or the immunoglobulin receptor. A region of the BBB is stimulated to improve delivery of the immunoglobulin receptor across the BBB at the region. The exosome is introduced to the region of the BBB, and the immunoglobulin receptor from the exosome is exposed to neural tissue of the patient, for example after crossing the BBB.

The exosome is generally derived from cell lines containing receptors for immunoglobulins, for example a white blood cell, a lymphocyte, a monocyte, a T cell, a B cell, a dendritic cell, a neutrophil, a basophil, an eosinophil, a stem cell, or an immune cell progenitor. The exosome can otherwise be cultured or derived from an immune cell line, derived from a cell line that naturally includes an immune receptor (e.g., Ig receptor), derived from a cell line engineered to include an immune receptor, or the exosome is otherwise derived in a way to include an immune receptor. Generally the exosome comprises an antibody receptor. In some embodiments the exosome is modified to improve adhesion of the exosome with the BBB, to express an antibody receptor, to release its contents across the BBB, or to traverse the BBB without tearing or otherwise disrupting the BBB.

Exposing the immunoglobulin receptor to neural tissue entails adhering or improving the adhesion of the exosome to the BBB. Alternatively or in combination, the exosome releases the immunoglobulin receptor across the BBB without traversing the BBB, or the exosome traverses the BBB and subsequently releases the an immunoglobulin receptor. It is contemplated the exosome can be engineered or otherwise modified to enable or improve methods of exposing the immunoglobulin receptor to neural tissue.

In some embodiments the immunoglobulin receptor is biologically active or activated when loaded into the exosome, though the immunoglobulin receptor can also or alternatively be active or activated when the exosome or receptor is introduced to the region of the BBB, or when the receptor is exposed to neural tissue. The immune agent includes at least part of an immunoglobulin receptor, an antibody, an immunoglobulin, an antigen, an antigen binding site, or other immune activating or modulating element. The immune agent or immunoglobulin receptor can be pre-loaded into the exosome, or can be loaded into the exosome by at least one of electroporation, incubation, sonication, extrusion, or transfection.

Stimulating the region of the BBB can be accomplished by directing focused energy at the region. For example, directing focused energy can include one or more of directing focused ultrasound, transcranial magnetic stimulation, shockwave, or light therapy, alternatively, in sequence, or other combination.

The exosome can be administered to the patient intravenously, intramuscularly, intrathecally, subcutaneously, orally, inhaled, or transdermally. The exosome can be loaded with the immunoglobulin receptor before stimulating the region of the BBB or can be pre-loaded with the agent.

Methods of delivering an immunoglobulin (Ig) across the BBB of a patient are contemplated. An exosome having (e.g., carrying, embedding, encasing, expressing, etc.) the immunoglobulin is administered to the patient. Focused ultrasound is applied to a region of the BBB, and the immunoglobulin is exposed to neural tissue of the patient. The Ig can be exposed or applied to the region by the exosome traversing the BBB at the region and releasing the immunoglobulin. Alternatively or in combination the exosome can release the immunoglobulin through the BBB without the exosome itself passing through the BBB.

The exosome is typically derived from one of a white blood cell, a lymphocyte, a monocyte, a T cell, a B cell, a dendritic cell, a neutrophil, a basophil, an eosinophil, a stem cell, or an immune cell progenitor. The exosome can be modified to improve adhesion of the exosome with the BBB, express an antibody receptor, release its contents across the BBB, or traverse the BBB without tearing or disrupting the BBB.

In some embodiments exposing the immunoglobulin to neural tissue entails adhering the exosome to the BBB using a receptor of the exosome, whether native to the exosome or engineered. Another focused ultrasound, a transcranial magnetic stimulation, a shockwave, or a light therapy can further be applied to the region of the BBB to aid delivery of the Ig. Preferably focused ultrasound is applied without use of microbubbles at the region.

It is contemplated that receptors are expressed by the exosome or otherwise present at the surface of the exosome to enable or improve crossing the BBB. For example, immune cell lines used to generate the exosomes or to otherwise modify the exosomes to have receptors aiding in adhering to or passing through the BBB. Such exosomes can be prepared by culturing monocytes and separating cell contents, for example by centrifuge. Immune cell progenitors and other stem cells can be used, or exosomes having an antibody receptor can be used, whether naturally occurring or engineered for this receptor capability.

In some embodiments, exosomes derived or cultured from monocytes are loaded with antibodies or Ig. Such exosomes exhibit increased adhesion to the BBB or otherwise cross the BBB without tearing or disruption. Thus, exosomes are contemplated as a delivery mechanism for an antibody.

As antibodies do not generally cross the BBB, even when therapeutically intended to, receptors from white blood cells, monocytes/leukocytes, dendritic cells, and similar immune system cells can be used with exosomes to improve delivery at the BBB. Exosomes derived from these types of cells (either allogeneic/autologous, conditioned/cultured) will also contain antibody receptors. Antibodies can be added/pre-loaded into an exosome injectate, which could then be administered intravenously, intramuscularly, intrathecally, subcutaneously, or even inhaled orally, as a way of facilitating improved delivery across the BBB.

Use of focused energy techniques, for example ultrasound, TMS, shockwave, or light therapy, can be used to improve or enable of delivery of modified exosomes, or Ig or antibody contents thereof, across the BBB.

Viewed from another perspective, monocytes (and their exosomes) have receptors that allow antibodies to adhere to the BBB. Exosomes may get across the BBB without microbubbles using targeted low-intensity focused ultrasound, specifically for the delivery of antibodies across the BBB.

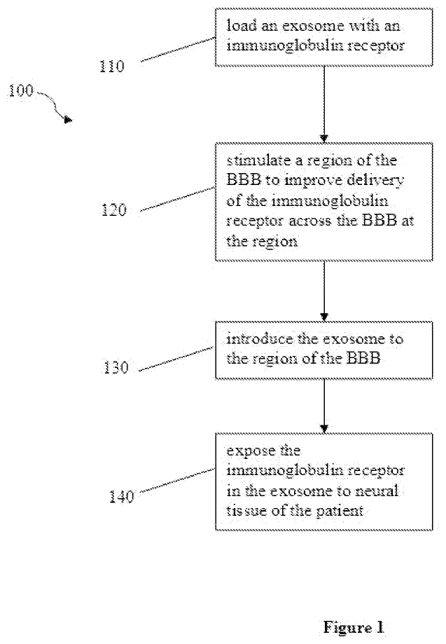

FIG. 1 depicts method 100 of the inventive subject matter, including ordered steps 110, 120, 130, and 140 in sequence. It is preferred the steps of the method are performed in the order depicted, though it is contemplated steps 110, 120, and 130 can be reordered in some embodiments.

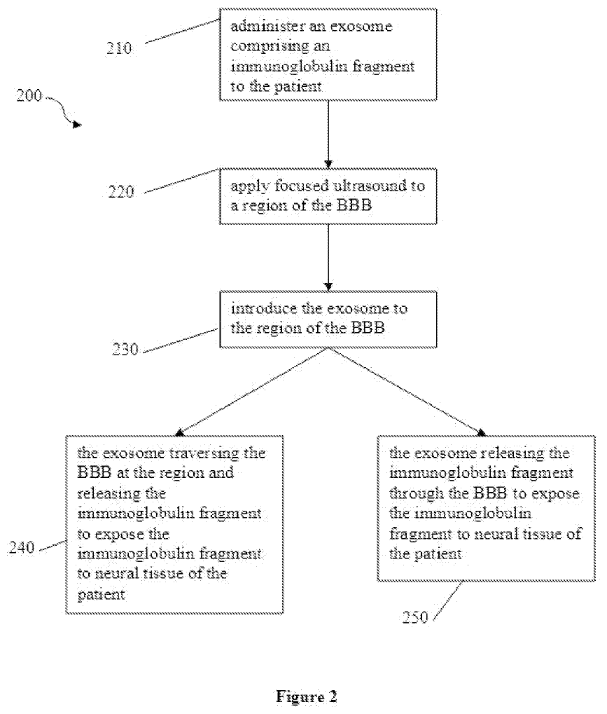

FIG. 2 depicts method 200 of the inventive subject matter, including ordered steps 210, 220, and 230 in sequence, with alternative steps 240 and 250. It is preferred the steps of the method are performed in the order depicted, though it is contemplated steps 210, 220, and 230 can be reordered in some embodiments. It is preferred that either alternative step 240 or alternative step 250 is performed in an embodiment, though some embodiments are contemplated including both steps 240 and 250.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of delivering an immunoglobulin receptor across a blood-brain barrier (BBB) of a patient, comprising:
loading an exosome with the immunoglobulin receptor;
stimulating a region of the BBB to improve delivery of the immunoglobulin receptor across the BBB at the region;
introducing the exosome to the region of the BBB; and
exposing the immunoglobulin receptor in the exosome to neural tissue of the patient.

2. The method of claim 1, wherein the exosome is derived from one of a white blood cell, a lymphocyte, a monocyte, a T cell, a B cell, a dendritic cell, a neutrophil, a basophil, an eosinophil, a stem cell, an immune cell progenitor, or a cell altered to comprise a second immunoglobin receptor.

3. The method of claim 1, wherein the exosome is cultured from an immune cell line.

4. The method of claim 1, wherein the exosome comprises an antibody receptor.

5. The method of claim 1, wherein the exosome is modified to improve adhesion of the exosome with the BBB, express an antibody receptor, release its contents across the BBB, or traverse the BBB without tearing the BBB.

6. The method of claim 1, wherein the step of exposing the immunoglobulin receptor to neural tissue comprises one of adhering the exosome to the BBB, the exosome releasing the immunoglobulin receptor across the BBB without traversing the BBB, or the exosome traversing the BBB and subsequently releasing the immunoglobulin receptor.

7. The method of claim 1, wherein the immunoglobulin receptor is biologically active when loaded into the exosome, when the exosome is introduced to the region of the BBB, or when the immunoglobulin receptor is exposed to neural tissue.

8. The method of claim 1, wherein the immunoglobulin receptor comprises at least part of an antibody, an immunoglobulin, an antigen, or an antigen binding site.

9. The method of claim 1, wherein the immunoglobulin receptor is loaded into the exosome by at least one of electroporation, incubation, sonication, extrusion, or transfection.

10. The method of claim 1, wherein the step of stimulating the region of the BBB comprises directing focused energy at the region.

11. The method of claim 10, wherein directing focused energy comprises one of directing focused ultrasound, transcranial magnetic stimulation, shockwave, or light therapy.

12. The method of claim 1, wherein the exosome is administered to the patient intravenously, intramuscularly, intrathecally, subcutaneously, orally, or inhaled.

13. The method of claim 1, wherein the exosome is loaded before stimulating the region of the BBB.

14. A method of delivering an immunoglobulin fragment across the blood-brain barrier (BBB) of a patient, comprising:
administering an exosome comprising the immunoglobulin fragment to the patient;
applying focused ultrasound to a region of the BBB;
exposing the immunoglobulin fragment to neural tissue of the patient by (i) the exosome traversing the BBB at the region and releasing the immunoglobulin fragment or (ii) the exosome releasing the immunoglobulin fragment through the BBB.

15. The method of claim 14, wherein the exosome is derived from one of a white blood cell, a lymphocyte, a monocyte, a T cell, a B cell, a dendritic cell, a neutrophil, a basophil, an eosinophil, a stem cell, an immune cell progenitor, or a cell altered to comprise an immunoglobin receptor.

16. The method of claim 14, wherein the exosome is modified to improve adhesion of the exosome with the BBB, express an antibody receptor, release its contents across the BBB, or traverse the BBB without tearing the BBB.

17. The method of claim 14, wherein the step of exposing the immunoglobulin fragment to neural tissue further comprises adhering the exosome to the BBB using a receptor of the exosome.

18. The method of claim 14, further comprising applying at least one of a second focused ultrasound, a transcranial magnetic stimulation, a shockwave, or a light therapy to the region of the BBB.

19. The method of claim 14, wherein the focused ultrasound is applied without use of microbubbles.

20. The method of claim 14, wherein the immunoglobin fragment comprises one of an immunoglobin receptor or a whole immunoglobin.
